(19) United States
(12) Patent Application Publication
(10) Pub. No.: US 2013/0131688 A1
(43) Pub. Date: May 23, 2013

(54) TWO-STAGE SNARE-BASKET MEDICAL DEVICE

(71) Applicant: Jeremy Schwartz, New York, NY (US)

(72) Inventor: Jeremy Schwartz, New York, NY (US)

(21) Appl. No.: 13/745,563

(22) Filed: Jan. 18, 2013

Related U.S. Application Data
(63) Continuation-in-part of application No. 11/982,245, filed on Oct. 31, 2007.
(60) Provisional application No. 60/894,022, filed on Mar. 9, 2007.
(60) Provisional application No. 61/588,889, filed on Jan. 20, 2012.

(51) Int. Cl. A61B 17/221

(52) U.S. Cl. CPC A61B 17/221
USPC 606/113

(57) ABSTRACT

The present invention relates to a medical device which can be used to resect and remove polyps, stones or foreign bodies. It accomplishes this goal by using a variable configuration basket which may act as a snare and a maneuverable basket. The various wires of the variable configuration basket are controllable by the operator.

### CROSS REFERENCE

The present application is a Continuation-In-Part of U.S. patent application Ser. No. 11/982,245 filed Oct. 31, 2007, which claims the benefit of U.S. Provisional Application No. 60/894,022 filed Mar. 9, 2007; each of which is incorporated herein by reference in its entirety. The present application also claims the benefit of U.S. Provisional Application Ser. No. 61/588,889 filed Jan. 20, 2012, which is incorporated herein by reference in its entirety.

### FIELD OF THE INVENTION

The present invention relates generally to medical devices. More particularly, this invention relates to a medical device for use in endoscopy and polypectomy that performs the dual functions of both a severing or resection of a gastrointestinal (or “GI”) polyp from the gastrointestinal tract wall and a trapping and retrieval of the gastrointestinal polyp from the gastrointestinal tact. Alternatively, this invention may also perform as a pure medical retrieval device of polyps, foreign bodies or stones.

### BACKGROUND OF THE INVENTION

Colorectal cancer is the third most common type of cancer in the U.S. In 2007 it is estimated that over 153,000 people will be diagnosed with colorectal cancer and over 52,000 people will die of this disease, making it the number two cancer killer in the United States. Colorectal cancers almost universally must be removed surgically, and a significant portion of patients with colorectal cancer will also require chemotherapy and radiation therapy.

The overwhelming majority of colorectal cancers (about 85-90%) develop from pre-cancerous (adenomatons) colon polyps. Colonoscopy is a medical procedure in which almost all colorectal polyps (big and small) can be found and removed by a technique known as polpectomy.

In a colonoscopy, a colonoscope (a long, flexible tube with a lens and a light source) is inserted into the anus in order to visualize the entire colorectum. The colonoscope contains a long channel through which the doctor can pass various medical devices for both diagnostic and therapeutic purposes. The diameter of this channel usually ranges from 2.8-4.2 mm depending on the manufacturer and specific model of colonoscope being used.

Current guidelines recommend that all colorectal polyps be completely removed and sent for a pathologic evaluation to determine if they are pre-cancerous or cancerous. Polyp removal is a 2-stage process. First, the polyp is resected or severed from the colorectal wall. Second, the severed polyp is retrieved from the inside of the colorectum.

Colonoscopy with complete removal of all polyps is the most effective way to reduce the incidence and death rates from colorectal cancer. Reductions in the incident rate of colorectal cancer is reported to be as high as 90% when the techniques of colonoscopy and polypectomy are properly utilized.

A colonoscopy is recommended for healthy people over 50 years of age; healthy people under 50 years of age with a family history of colorectal cancer; people with blood in their stool, a change in their bowel habits, or significant abdominal pain; and people who are iron-deficient which might indicate slow blood loss from the digestive tract. Approximately 80% of adult colonoscopies in the U.S. area performed on patients over 50 years of age.

In 1999, approximately 4.4 million colonoscopies were performed in the U.S. That number is now likely closer to 5 million per year due to increased public awareness (i.e., Katie Couric) and more endoscopists graduating from GI training programs in recent years. According to the CDC, however, about 41.8 million people aged 50 or older in the U.S. have not undergone screening for colorectal cancer as recommended by current guidelines.

Precancerous colorectal polyps are found in about 25-30% of patients over age 50 undergoing routine screening (i.e., no symptoms). Polyp size can vary from 1-2 mm all the way to greater than 6 cm. The probability of finding cancer within a colorectal polyp increases with increasing size.

Diminutive colorectal polyps are 1-5 mm in size. They have the lowest risk of being pre-cancerous or cancerous. Presently, resection and retrieval of diminutive colorectal polyps are accomplished using a single medical device, a biopsy forceps, which is removed from the colonoscope with the polyp trapped in the jaws of the forceps.

Small colorectal polyps are 6-9 mm in size. They present a low cancer risk and an intermediate risk of being pre-cancerous. They are usually resected with a snare loop (with or without electric current). Colorectal polyps 8 mm in size or less are usually retrieved via suctioning the specimen through the colonoscope into a specialized collection device. A significant number of these small polyps are lost at some point after entry into the colonoscope and are never retrieved.

Large colorectal polyps are greater than or equal to 1 cm in size. About 20% of colorectal polyps are in this category. The majority of these polyps are at least pre-cancerous. The risk of cancer increases directly with size. Resection is achieved with a snare loop (almost universally with cautery). Due to their size, they are too large to be suctioned into the colonoscope. As such, retrieval is more difficult and almost universally requires a medical device for assistance.

As mentioned above, about 41.8 million people in the U.S. currently go unscreened. Screening rates area likely to rise significantly in the near future as other, highly accurate but less invasive screening tests for colorectal cancer and polyps become commercially viable. The additional large colorectal polyps identified by these tests which will need to be removed by colonoscopy will likely number in the millions.

In addition to colorectal polyps, polyps can also occur in the upper gastrointestinal tract. Although there are fewer polyps in the upper GI tract then in the colorectum, their number is not insignificant. About 50,000-100,000 large upper GI polyps are removed annually. A significant percentage of these polyps are pre-cancerous and they can often cause abdominal pain and bleeding. Mechanisms for removal of these polyps are similar to those methods used to remove colorectal polyps.

Currently, there are a number of methods used to retrieve GI polyps, especially large polyps. Six of the more common methods are piecemeal retrieval, suctioning the polyp to the tip of an endoscope, use of the snare as a retriever, use of a grasper device, use of a basket device, and use of retrieval net. Each method has its disadvantages. It is estimated that between 5.7% and 16.5% of resected colorectal polyps are never retrieved. In addition to patient dissatisfaction, this can lead to misdiagnosis as well as the performance of unnecessary surgeries and attendant increases in morbidity and mortality. An additional percentage of polyps which are retrieved require significant increases in time to do so under the current art.

Piecemeal retrieval involves chopping the polyp up into pieces small enough to be suctioned through the endoscope. This occasionally occurs out of necessity with large, flat polyps which cannot be resected in one piece. A pathologist, however, will be unable to determine if resection is complete. Thus, the colonoscopy will often need to be repeated earlier then planned to re-inspect the polypectomy site for residual polyp tissue. Also, part of the specimen (possibly containing early cancer) may not be retrieved.

The suctioning of a polyp to an endoscope tip may work adequately for some rectal polyps. There is, however, a significant risk of the polyp being dropped as suction is inadequate to reliably secure the polyp. A dropped polyp can be lost or (in the case of upper GI tract polyps) can lead to a catastrophic outcome if it is dropped into the trachea.

The use of a snare to retrieve polyp is an attractive option to many endoscopists. It saves time needed to change devices and saves money because there is no added cost in using the same device to retrieve as well as resect the polyp. Snares cost about ten to twenty dollars each. A snare, however, is two dimensional. Thus, while the polyp is easily acquired, it is not secure and can be easily dropped. In addition, the snare can inadvertently bisect the polyp, leading to difficulty in pathologic interpretation as well as the loss of part or all of the polyp.

Graspers usually have three prongs, although four or five pronged graspers are available. Graspers decrease the rate at which specimens are dropped, although some specimens are still dropped because of the grasper's open distal design. Also, the grasper is a second device, different from the snare used for resection, and deploying the grasper adds time to the procedure and increases the chance of losing the specimen during this time interval. Graspers also add costs (about seventy-five dollars) to the procedure.

When using a basket device, the specimen is secure if it is acquired properly. However, it is harder to acquire very large polyps due to the basket's small size. The basket is also a second device whose deployment adds time to the procedure and risks the specimen being lost. There is also an added cost of over two hundred dollars each for using a basket.

Retrieval nets make it easier to acquire a specimen but the acquisition is less secure because the nets can tear. Also nets can be used to acquire multiple specimens. Again, however, as is the case with basket devices and graspers, the nets are a second device requiring time to deploy them. This creates a risk that a specimen can be lost. Also, the use of baskets increases the cost of a procedure by about seventy-five to eighty-five dollars.

Accordingly, there is a need for an improved polyp removal device for removal of small and large GI polyps.

There is also a need for an improved polyp removal device for removal of small and large GI polyps which makes it easy to acquire and retrieve a specimen, such as a resected polyp.

There is a further need for an improved polyp removal device for removal of small and large GI polyps which decreases the chance that a specimen can be lost.

There is a still further need for an improved polyp removal device for removal of small and large GI polyps which does not require the deployment of a second device which can, among other things, increase the time a procedure takes and increase the possibility that a specimen can be lost.

There is yet another need for an improved polyp removal device for removal of small and large GI polyps which does not increase the cost of a procedure.

There is yet another need for an improved endoscopic basket device with variable configurability of the basket wires to improve the acquisition of stones, polyps, and other objects as well as to account for larger and odd shaped stones, polyps, and other objects.

### OBJECTS OF THE INVENTION

It is an object of this invention to provide for an improved polyp removal device for removal of small and large GI polyps.

It is also an object of this invention to provide for an improved polyp removal device for removal of small and large GI polyps which makes it easy to acquire and retrieve a specimen, such as a resected polyp.

It is a further object of this invention to provide for an improved polyp removal device for removal of small and large GI polyps which decreases the chance that a specimen can be lost.

It is a still further object of this invention to provide for an improved polyp removal device for removal of small and large GI polyps which can both resect and retrieve a polyp and as such does not require the deployment of a second device.

It is yet another object of this invention to provide for an improved polyp removal device for removal of small and large GI polyps which does not increase the cost of a procedure.

In addition to the above, it is an object of this invention to provide for a medical basket retrieval device with improved acquisition and capture capabilities which can be used to retrieve other resected polyps, foreign bodies, stones and the like.

These and other objects of the invention are satisfied by the invention described more fully below.

### SUMMARY OF THE INVENTION

The present disclosure relates to a medical device for use in endoscopy and polypectomy. The device performs the dual functions of resection and retrieval with decreased possibility that a specimen will be lost or damaged. In the alternative, the present device can be used to retrieve other polyps, foreign bodies, stones and the like.

The present device functions by use of a novel variable configuration basket. This and other embodiments of the present device are discussed more fully below.

### DESCRIPTION OF THE DRAWINGS

FIG. 1A shows a top view of the inventive device in a closed position.

FIG. 1B shows a top view of the inventive device in an open position, i.e. with the snare deployed.

FIG. 2A shows a view of the distal end of the inventive device in the open position where the snare is surrounded by one type of sheath and is a first snare configuration.

FIG. 2B shows a view of the distal end of the inventive device in the open position where the sheath is withdrawn and the device is in a second basket configuration.

FIG. 3A shows a view looking at the distal end of the inventive device, where the wires of the snare are one on top of the other and in an undeployed configuration, i.e. a first snare configuration.

FIG. 3B shows a view of the FIG. 3A device which is in its deployed or second basket configuration.

FIG. 3C shows a view similar to that of FIG. 3A, where the snare wires cross over each other to form an X configuration at their distal point, shown here as a cup.

FIG. 3D shows a view of the FIG. 3C in its deployed or second basket configuration.

FIG. 3E shows a view looking at the distal end of the inventive device, where the wires of the snare are configured one on top of the other and in an undeployed configuration, i.e. a first snare configuration.

FIG. 3F shows a view of the FIG. 3E device which is in its deployed or second basket configuration.

FIG. 3G shows a view looking at the distal end of the inventive device, where the wires of the snare are configured one on top of the other and cross over each other to form an X configuration at their distal end, and which is in an undeployed configuration, i.e. a first snare configuration.

FIG. 3H shows a view of the FIG. 3G device which is in its deployed or second basket configuration.

FIG. 4A through FIG. 4E shows one embodiment of the present device in use to resect and retrieve a polyp.

FIG. 5A shows a top view of an alternate embodiment of the inventive device in a closed position.

FIG. 5B shows a top view of an alternate embodiment of the inventive device in an open two-dimensional position, i.e., with the variable configuration basket deployed in a snare-type configuration.

FIG. 6 shows a view of the distal end of an alternate embodiment of the inventive device in the open position where the sheath is withdrawn and the device is in a second basket configuration.

FIGS. 7A-7D show views of the distal end of the inventive device, with the wires of the variable configuration basket deployed in the basket configuration. The arrows indicate possible movement of basket wires.

FIG. 8 shows a perspective view of an alternative embodiment of the inventive device in an open position, i.e., with the basket deployed.

FIG. 9 shows a top view of an alternate embodiment of the inventive device in an open position, i.e., with the basket deployed.

FIG. 10 shows a top view of an alternate embodiment of the inventive device in a partially open position, i.e., with a portion of the basket deployed.

FIGS. 11A-11D shows views looking at the distal end of the inventive device, where the wires of the variable configuration basket device which is in its deployed or partially deployed configuration indicating movement of individual basket wires.

FIG. 12 shows a cut away view of a tubular catheter in an embodiment of the inventive device with a lithotripter device.

### DETAILED DESCRIPTION OF THE INVENTION

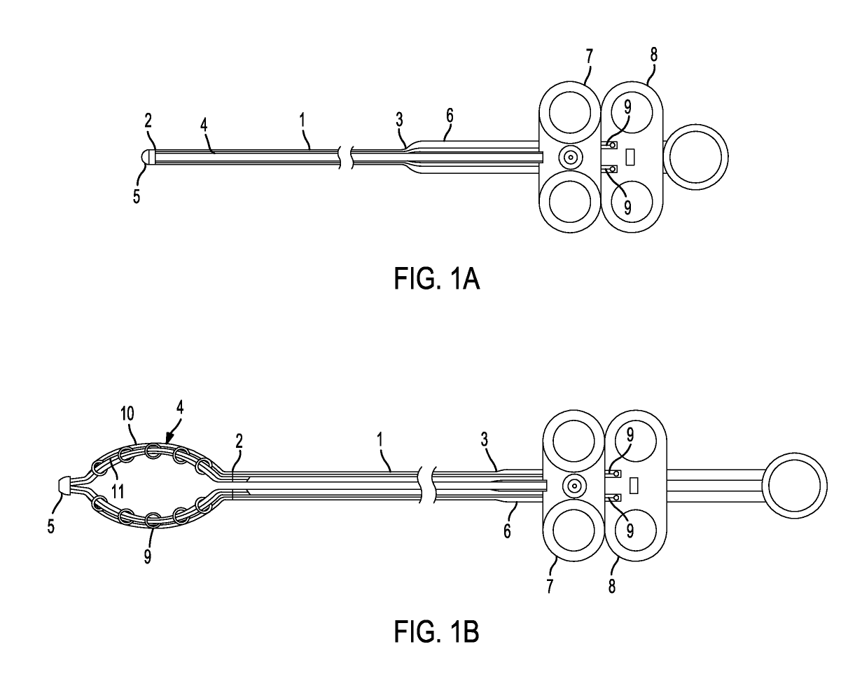

In a preferred embodiment, the present invention, referred to herein as the “Two-Stage Snare-Basket,” is a medical device that can be passed through a colonoscope, upper gastrointestinal endoscope, duodenoscope or an enteroscope and operated during a colonoscopy, upper gastrointestinal endoscopy (esophagogastroduodenoscopy), ERCP (endoscopic retrograde cholangio-pancreatography) or enteroscopy procedure, respectively. The Two-Stage Snare-Basket comprises a handle on one end of an elongated tubular element that is connected to and controls a first snare configuration on the other end in the first stage of operation that can be converted into a second basket configuration in the second stage of operation. Initially, the first snare configuration is in a closed position, as shown in FIG. 1A. It can be converted to the open or first snare position, as shown in FIG. 1B, by for example, pushing on a handle (such as (7), (8) of FIG. 1A and FIG. 1B, described in more detail below). Stored energy in the snare will cause it to assume its first snare position. In an alternate embodiment, the snare can spring out of its closed position to its first snare configuration using pre-loaded energy, such as a spring. It should be noted that by whatever means is employed to convert the snare from the closed position to the open position, all of the snare wires, be they full snare wires or half snare wires (discussed more fully below), preferably extend simultaneously.

The first snare configuration in the first stage functions as a resection snare with or without electrocautery to sever the polyp (or portion of an extremely large polyp in a multi-step breakup and removal process) from the GI tract wall and initiates acquisition of the resected polyp (or severed portion of an extremely large polyp). The first snare configuration is easily converted into the second basket configuration for the second stage by control of the handle. The second basket configuration functions in the second stage by control of the handle as a polyp trapping or retrieval element to remove the severed polyp (or severed portion of an extremely large polyp in a multi-step process) from the inside of the GI tract.

In a preferred embodiment, the first snare configuration is comprised of two or three (or more) snares that are juxtaposed on top of one another and/or inside of one another and are held in snare configuration by a stored energy mechanism. Each of the two or three (or more) snares may be comprised of a whole snare (one looped wire each) or two half-snares (two wires that are connected in and/or held by a distal cap) that run from the handle end through the elongated tubular element to a distal cap that permits flexibility of movement of the snare wires between the first snare configuration and the second basket configuration. In this embodiment, the change from the first snare configuration to the second basket configuration is effected via the release of stored mechanical energy at or just proximal to the site of the snare basket itself via a detent mechanism. One stored energy mechanism can be a sheathing element which consists of a wire that holds the snare wires together and runs parallel to the snare wires to the handle end. The sheathing element can be in the form of rings, or alternatively a solid sheath, or spring-loaded hooks. It is understood, however, that while this embodiment and other embodiments of the present device employ a detent mechanism, the present device is not limited to the use of detent mechanisms only.

In an embodiment, the first snare configuration is designed such that it can be looped over to surround the polyp in the extended configuration. The first snare configuration can then be tightened by operation of the handle and then at least one of the snares in the first snare configuration is operated as a cauterization tool. The snare with a cautery function will be attached to a source of electricity through an electrocautery snare plug or other mechanism located within the device handle. Following cauterization and resection of the polyp, the snare configuration can then be held in position surrounding the resected polyp (or severed portion of an extremely large polyp).

The first snare configuration of the embodiment is easily converted by control of the handle to the second basket configuration by retracting or releasing the sheathing element that holds the various snare wires together during polyp retrieval. When the sheathing element is retracted, the two snares spring or rotate apart and assume the second basket configuration.

Following conversion of the first snare configuration into the second basket configuration, the second basket configuration can be tightened around the resected polyp (or severed portion of an extremely large polyp) by control of the handle to securely trap the resected polyp (or severed portion of an extremely large polyp) for retrieval.

In various embodiments of the invention, the size and shape of the snare and basket may vary, angulation of wires in the basket configuration may vary, and the length and the diameter of the medical device and the elongated tubular element may also vary.

In various embodiments of the device, the number of snares within the first snare configuration (and accordingly the number of wires in the corresponding second basket configuration) may vary and whether the snares consist of whole snares or half-snares may vary. The arrangements and connectivity of the half-snares and whole snares may also vary. In addition, whether one or more of the snares or the sheathing element functions as the cauterization tool may vary. The methods or mechanism of conversion from the first snare configuration to the second basket configuration may also vary.

In various embodiments of the device, the sheathing element may vary. The sheathing element can be in the form of rings, spring-loaded hooks or a solid sheath. In other embodiments of the device, there may not be a sheathing element, but instead, the device may contain a rotational or flip lever detent mechanism which does not utilize the release of stored energy to effect the conversion from the first snare configuration to the second basket configuration.

In various embodiments of the device, the handle that controls the first snare configuration and second basket configuration and conversion between the two configurations may vary. The handle may be comprised of a single handle with two or more levers and a locking device or may be a combination of any number of handles, any number of levers, and any number of locking devices. A locking device may not even be necessary in various embodiments. The location and design of the electrocautery plug may also vary. The handle may also contain various configurations of rotatable thumbwheels, dials or knobs to control rotation of individual or groups of basket wires.

This present device has the potential to save time and lower costs in that one single device will perform both resection and retrieval of polyps rather than two separate devices. For instance, in removal of large colorectal polyps in particular, resection or severing is usually accomplished by one device and trapping and retrieval is usually accomplished by a second device.

This device may also simplify the retrieval of large polyps, which can often be more difficult to trap and retrieve due to polyp size and shape, and if extremely large, may require a multi-step breakup and removal. In the first snare configuration, for instance, the open two-dimensional design at the initial stage of acquisition provides a larger space than the narrow space between the wires in prior basket devices. This device is also capable of repeated uses to resect and retrieve multiple polyps (or multiple portions of an extremely large polyp) from the same patient during a single colonoscopy procedure. This device may also simplify the removal of small polyps by achieving a method for complete pull-through-the-endoscope removal of small polyps analogous to how biopsy forceps provide a method for complete pull-through-the-endoscope removal of diminutive polyps. The device may also provide for more accurate control and retrieval of polyps of irregular size and shape.

This device may also function as a pure retrieval device for already severed polyps (or severed portions of extremely large polyps), foreign bodies and stones in the gastrointestinal tract, or in other organs or body cavities. For use as a pure retrieval device, the device would not require electrocautery function and the corresponding electrocautery elements of the device for resection of a polyp would not be required.

In fact, the present device has numerous applications, and represents a major improvement in specimen acquisition for basket retrieval technology. For polyp removal, it can be used in the gastrointestinal tract, such as the colorectal, small intestinal, gastric and esophageal areas. For gynecological polyps, it can be used in the uterer, cervix and vagina. The disclosed device can also be used to remove polyps in the genitourinary tract, such as the bladder and urethra. The disclosed device is also useful in removing ear, nose and throat polyps, such as nasal, palatal, oral and vocal cord polyps. In addition, the present device can be used to remove polyps in the peritoneal cavity.

The present device can also be used for stone removal. This includes stones in the gastrointestinal tract, such as the biliary tree (intra-hepatic and extra-hepatic), pancreas and gallbladder. It can also be used in the genitourinary tract to remove stones in the kidney, bladder and ureter.

Furthermore, the disclosed device is useful in foreign body retrieval. For example, the disclosed device can remove foreign bodies in the gastrointestinal tract, such as colorectal, gastric, esophageal and small intestinal foreign bodies. It can remove foreign bodies from the genitourinary tract, such as from the urethra and bladder. It can be used for gynecological foreign body removal, such as in the vagina, cervix and uterus. It can be used to remove intra-abdominal or intra-peritoneal foreign bodies, such as during laparoscopic gallbladder procedures. Furthermore, it can be used to remove foreign bodies from the thoracic cavity, such as debris, medical instruments and resection specimens.

Turning now to the drawings, FIG. 1A shows the inventive device in the closed position, i.e. where the snare (4) is not deployed. The device comprises an elongated tubular element or a tube (1) with a distal end (2) and a proximal end (3). The tube (1) is preferably flexible and can be made of, for example, catheter based plastic.

The tube (1) preferably has an outside diameter from about 2.8 mm to about 4.2 mm, more preferably about 2.8 mm to about 3.7 mm.

The tube (1) contains a snare (4) at its distal end (2), which terminates in a cap (5).

The proximal end (3) of the tube (1) is connected to a control unit or a grip (6). The grip (6) is preferably less flexible then the tube (1). The grip (6) can be made of, for example, plastic or metal.

A first slidable handle (7) and a second slidable handle (8) are slidably disposed on the grip (6). There is an optional connection means (not shown) which connects the first slidable handle (7) to the second slidable handle (8). That way, the two handles (7, 8) can move in unison. The connecting means is disengageable so that the slidable handles (7, 8) can be moved independently of each other.

FIG. 1B shows the device with the snare (4) extended or deployed, i.e. in a first snare configuration. Part of the snare (4) is a sheath (9) which surrounds a first snare wire (10) and a second snare wire (11). The snare wires (10, 11) are held together by the sheath (9). Also, the sheath (9) extends through the tube (1), into the grip (6) and attaches to the second slidable handle (8). The snare wires (10, 11) also pass through the tube (1) and into the grip (6) to attach to the first slidable handle (7) via a means described more fully below.

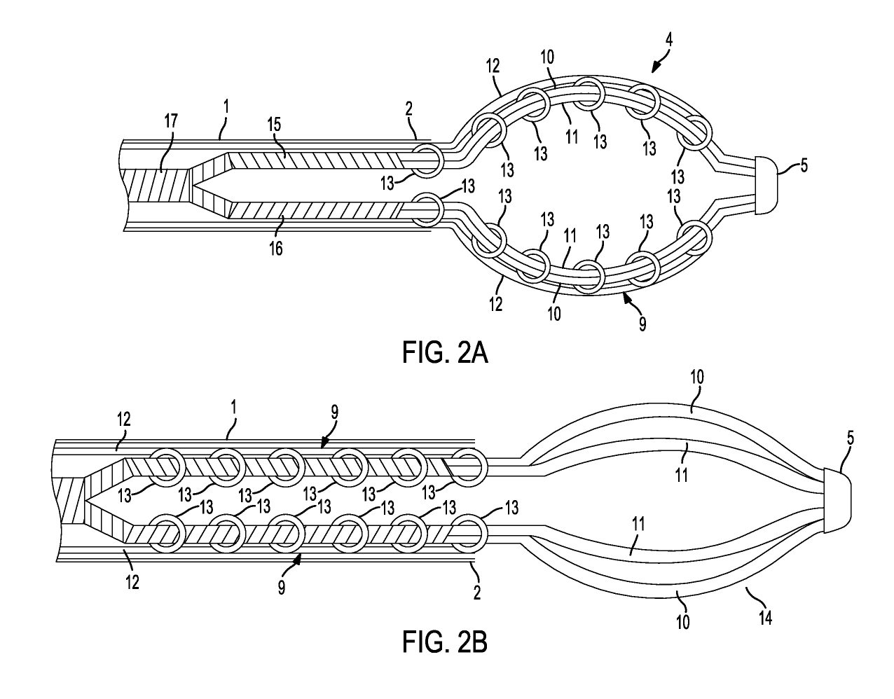

FIG. 2A and FIG. 2B show the snare (4) in more detail. FIG. 2A shows the snare (4) in a deployed or first snare configuration. It is surrounded by the sheath (9) which keeps the first snare wire (10) and the second snare wire (11) together. In the particular embodiment shown in FIG. 2A, the sheath (9) comprises a sheath backing (12) and a plurality of sheath rings (13). In this configuration, it is the sheath backing (12) which passes through the tube (1) into the grip (6) and connects to the second slidable handle (8), the attachment shown as (9) in FIG. 1A and FIG. 1B.

As shown in FIG. 2A and FIG. 2B, as well as FIG. 1B, the snare wires (10, 11) terminate in a cap (5). It is understood, however, that instead of a cap (5), one or both of the snare wires (10, 11) could form a continuous loop (not shown). The wires (10, 11) of the snare basket device (4, see also 14) at about, just proximal to, or at their distal ends can fuse into or attach to a single wire or braid into each other at about, just proximal to, or at, the most distal aspect of the snare basket device (4, see also 14), thereby forming the most distal aspect of the snare basket device. The distal end of the snare basket device (4, see also 14) is not left open, but rather the snare wires (10, 11) will connect to each other as described above either directly (braiding, fusion, etc.) or indirectly (via a cap (5) or intervening wire or wires, etc.).

As discussed above, it is understood that the sheath (9) can have various configurations. The configuration shown in, for example, FIG. 2A shows the sheath made of metal sheath rings (13) supported by a wire sheath backing (12). Alternatively, the sheath can be solid, such as a flexible metal member or flexible plastic or rubber member, or the rings can be partial rings, i.e. not fully closed, or spring loaded hooks or clips. Alternatively, pincers oriented from distal to proximal or proximal to distal can be used to hold the snare wires (10, 11) together. The pincers can be released, for example by a spring loaded means, or simply withdrawn into the tube (1) to allow the formation of the second basket configuration (14) as shown in FIG. 2B. Two to four pincers arraigned as up-down or left-right pairs may be present on each side of the snare (4).

It should also be noted that although FIG. 2B shows the sheath (9) being withdrawn proximally into the tube (1), in an alternative embodiment the sheath (9) can be pushed to the distal end of the snare (4).

The snare (4) as shown in FIG. 1B and FIG. 2A, or the second basket configuration (14) of FIG. 2B, is made of two complete snare wires (10, 11), although the device is not so limited. For example, there can be three, four or more snare wires used in the present device. Likewise, one or more (but not all) of the snare wires can be half wires. A half wire will also be connected to the cap (5) or otherwise connected to the other snare wires (10 or 11).

The snare wires (10, 11), can be flat wire, braided wire or shaped memory wire, or any suitable material which can function as a first snare configuration (4) and a second basket configuration (14).

Preferably, the snare wires (10, 11) are made of stainless steel, nickel titanium alloy (Nitinol) or titanium.

The snare wires (10, 11) as shown in FIG. 2A are adjacent to each other in the first snare configuration (4). Alternatively, from a top view, they can be juxtaposed to be one on top of the other. Within the tube (1), the snare wires (10, 11) first braid with each other to form first braids (15, 16) and those first braids (15, 16) are in turn braided together to form a second braid (17). This second braid (17) then passes through the tube (1) and into the grip (6) to attach to, in the embodiment shown in FIG. 1A and FIG. 1B, the first slidable handle (7).

It is understood that the braiding is not necessary to practice the present invention. For example, the snare wires (10, 11) can simply pass through the tube (1) and into the grip (6) to connect with the first slidable handle (7). An alternative embodiment can use different materials. For example, the actual portion of the snare wires (10, 11) which form the first snare configuration (4) and the second basket configuration (14) can be made of stainless steel, Nitinal or titanium. Then, in place of the second braid (17) or even the first braids (15, 16) the same or different material can be used as a single piece. Thus, this single piece can be stainless steel, titanium, nitinal, or a different metal, or even a non-metallic substance, such as plastic. The first braids (15, 16) or even the snare wires (10, 11) themselves can be attached to this single piece by conventional means, such as welding, soldering, clamps, glue and the like.

The formation of the second basket configuration (14) can occur in a number of ways. In one preferred embodiment, the snare wires (10, 11) are made of a shaped memory wire or otherwise configured so that in their resting or unconstrained state, they would assume the second basket configuration (14). They are held in their first snare configuration (4) by the constraining action of a detent mechanism utilizing stored energy. This detent mechanism might utilize, among other mechanisms, rings, hooks, pincers or sheathing. This detent mechanism might be effected by a spring or the manipulation of the handle in order to release the stored energy to effect the change in configurations. The spring can be located in the tube (1) and can be activated when desired, such as by pushing a button which will release the spring. In another embodiment, a torque can be applied to the snare wires (10, 11) to rotate them from the first snare configuration (4) to their second basket configuration (14). Torque can be applied by any conventional means. For example, the first slidable handle (7) may also be rotatable, the rotation providing torque. There may also be a torque means, such as a wheel or knob, which can be turned to provide torque. This torque means can be present, for example, somewhere on the tube (1) or grip (6), or can even be placed on the first slidable handle (7).

In an alternate embodiment (not shown) a support means can be provided at the distal end (2) of the tube (1). This support means prevents deformation, fraying or tearing at the distal end (2) of the tube (1). It can be in the shape of a ring or a cap and is made of any suitable material, such as plastic.

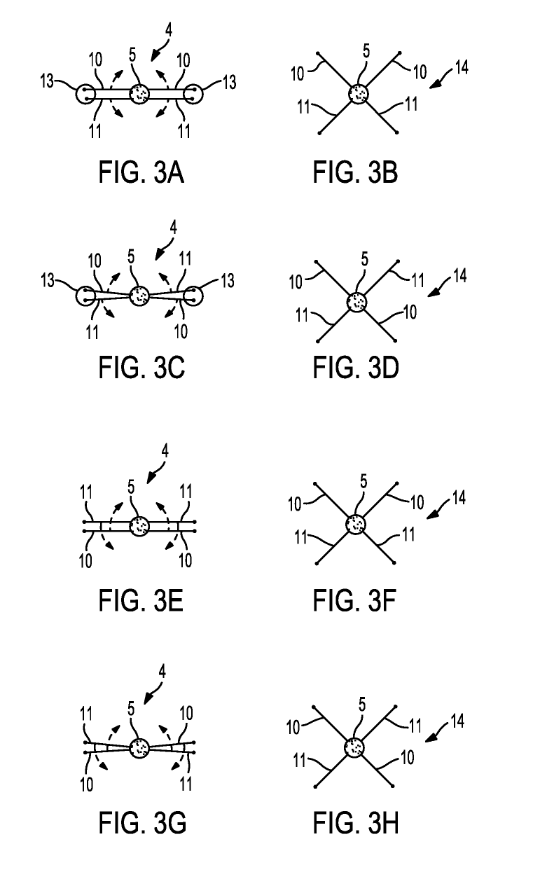

The configuration of the snare wires (10, 11) relative to each other can take several different configurations. These are shown in more detail in FIG. 3A through FIG. 3H, which shows just the snare wires (10, 11) and the cap (5) from a head-on perspective.

FIG. 3A is similar to the configuration of FIG. 2A. In both, one (or first) snare wire (10) is positioned outside the other (or second) snare wire (11) when in the first snare configuration (4). Rings (13) are also shown. When in the second basket configuration (14) of FIG. 2B and FIG. 3B, first snare wire (10) positions up and second snare wire (11) positions down, thus forming a basket. In FIG. 2B, first snare wire (10) would be positioned above the plane of the page and second snare wire (11) would be positioned below the plane of the page.

In an alternative embodiment, the snare wires (10, 11) of FIG. 3C are configured in a manner similar to FIG. 3A except that they cross over each other to form an X configuration at the cap (15). The snare wires (10, 11) move, as shown by the dotted lines in FIG. 3C, to form the second basket configuration (14) of FIG. 3D.

FIG. 3E shows an embodiment where the first snare wire (10) is under the second snare wire (11) in the first snare configuration (4). The top snare wire (11) has a downward force and the bottom snare wire (10) has an upward force. This holds the snare wires (10, 11) together in the first snare configuration (4) even without, for example, the rings (13) (not shown). When the snare wires (10, 11) are moved, so that they are no longer in contact with each other, the bottom snare wire (10) positions up and the lower snare (11) positions down form the second basket configuration (14) of FIG. 3F.

FIG. 3G shows the first snare wire (10) and the second snare wire (11) cross over each other at the cap (5) to form an X configuration when in the first snare configuration (4). In a manner similar to that of FIG. 3E, the upper snare wires (11 to the left of the cap (5), 10 to the right of cap (5)) have a downward force and the lower snare wires (10 to the left, 11 to the right) have an upward force. To achieve the second basket configuration (14) of FIG. 3H, the top portion of each snare wire (10, 11) positions up and the bottom portion of each snare wire (10, 11) position down, as shown in FIG. 3H. This can be done by, for example, moving the snare wires (10, 11) relative to each other, in a manner similar to that described for FIG. 3E and FIG. 3F, above.

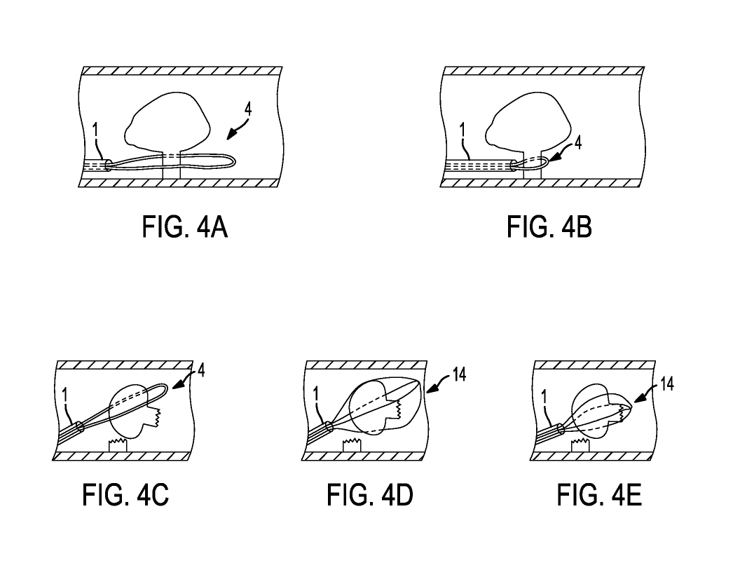

Of course, as described above, the cap (5) need not be present. Each snare wire (10, 11) can simply be a loop and be attached to each other at their distal ends. As shown in FIGS. 4A-4E, when in a basket configuration, the snare wires (10,11) may comprise non-parallel snares (4) each having a distal end extending beyond the distal end of the inner channel of the endoscope, wherein the at least two snares are overlapping or coupled at about or proximal to their distal ends.

As discussed above, and especially for polyp removal, the present invention can cauterize the polyp to resect it. A conventional source of electricity can be provided. In one embodiment, one or more of the snare wires (10, 11) performs the cautery function. In an alternate embodiment, this configuration may not be optimal because some of the cauterized sample may adhere to the snare wires (10, 11) and prevent or inhibit their deployment in the second basket configuration (14). To avoid this, the current for cauterization can pass through the sheath (9), which can be made, of a conducting material or contain a conducting material, such as a wire. To complete the circuit, the distal ends of the sheath (9) should be in contact or connected by, e.g., a wire or cap (5). In an alternative embodiment, current can pass through both the sheath (9) and one or more of the sheath wires (10, 11). Since only the sheath (9) will contact the sample for cauterization, little or no portion of the cauterized sample will contact any of the sheath wires (10, 11).

It should be noted that in an embodiment of the present invention a sheath is not used. For example, the snare wires (10, 11) may not have a shaped memory and when deployed in a first snare position (4) they will stay in close proximity to each other. Transition to the second basket configuration (14) can be effected by rotating one or both snare wires (10, 11) such as, for example, by applying torque, as previously described or through a detent mechanism utilizing for example flip levers or dials as previously described. In another embodiment, the snare wires (10, 11) are configured one on top of the other. The top snare wire (11) is made of shaped memory or is otherwise so configured to push down and the bottom snare wire (10) is configured to push up. These opposing forces hold the snare wires (10, 11) together in a first snare configuration (4). In alternate embodiments, where these snare wires (10, 11) contact each other can be a flat surface, a channel can be provided or a tongue and groove type system can be employed to more securely hold them in place. To convert to the second basket configuration (14) the snare wires (10, 11) can be simply moved relative to each other, i.e. front, back or side to side. Once the snare wires (10, 11) are no longer in contact with each other, they will snap into their second basket configuration (14). This is shown in, for example, FIG. 3E and FIG. 3F.

FIG. 4 shows one embodiment of the present invention used to resect and remove a colorectal polyp. As shown in FIG. 4A, once a polyp is located the first snare configuration (4) is used to surround the polyp. The snare (4) is then tightened and the polyp is cauterized, as shown in FIG. 4B. The first snare configuration (4) is then enlarged, if necessary, to surround the cauterized polyp, as shown in FIG. 4C. In FIG. 4D, the first snare configuration (14) is converted into the second basket configuration (14) by the means disclosed above. As shown in FIG. 4E, the second basket configuration (14) has been tightened around the resected polyp completing the highly secure acquisition process. The polyp is then removed, either by withdrawing the entire snare basket device out of the therapeutic channel of the endoscope (small polyp) or by withdrawing both the entire snare basket device and endoscope from the GI tract cavity in tandem (large polyp).

In another embodiment of the present invention, the sheath (9) can be used to control how many snare wires (10, 11) are released to form the second basket configuration (14). This may be necessary, for example, where space is limited. Referring to FIG. 2A and FIG. 2B, instead of withdrawing the entire sheath (9), only the left half or the right half is withdrawn. In effect, this forms a second basket configuration (14) with three wires instead of four wires.

As mentioned above, an endoscope includes a lens with a light source, as well as a means for viewing the acquired images. In addition, endoscopes include a therapeutic channel through which medical device accessories can be passed and whose diameter is usually between 2.8 mm and 4.2 mm. While not shown herein, it is understood that the present invention is compatible with these features.

In view of the above, and further in view of the drawings, especially FIG. 1A, FIG. 1B and FIG. 2B, the functioning of one embodiment of the present invention is apparent. An endoscope is inserted into the GI tract via the mouth, anus or a previously created surgical ostomy and is advanced to the desired segment of the GI tract via the operator. If a polyp is found, the inventive device is inserted into the therapeutic channel of the endoscope and is advanced through that therapeutic channel until its distal end extends out of the distal end of the therapeutic channel of the endoscope and into the GI lumen which contains the polyp. The first slidable handle (7) and the second slidable handle (8) are slid together in a distal direction, thus deploying the snare (4). The snare (4) is positioned around the polyp and pulled tight by pulling the first slidable handle (7) and second slidable handle (8) together in a proximal direction. Once properly positioned, an electric current is passed through the snare (4) to cauterize the polyp. The snare (4) can be enlarged again, if necessary, to surround the polyp by pushing the first slidable handle (7) and the second slidable handle (8) together in a distal direction. This also helps to align the polyp within the snare initiating polyp acquisition. Once the polyp is aligned, the second slidable handle (8) is pulled in a proximal direction, but the first slidable handle (7) is not moved. This pulls the sheath (9) down and off the snare wires (10, 11). Once the sheath (9) is removed from the snare wires (10, 11), they convert into the second basket configuration (14) because the sheath (9) is no longer holding them in place. This furthers the highly effective process of polyp acquisition. The second basket configuration (14) can then be closed as much as necessary in order to tighten the second basket configuration around the polyp to secure and complete acquisition of the polyp by pulling the first slidable handle (7) proximally until resistance to further sliding is felt indicating that the polyp is securely acquired within the second basket configuration (14). The second basket configuration (14) with the polyp secured can then be withdrawn through the endoscope (small polyp) or the entire endoscope assembly including the second basket configuration (14) with the polyp secured can be withdrawn (large polyp). Once removed from the body, the polyp is discharged from the second basket configuration (14). The sheathing (9) can be re-advanced over the snare wires (10, 11) either manually or by sliding the first slidable handle (7) and second slidable handle (8) together to reform the first snare configuration (4). The first snare configuration (4) is then closed, as shown in for example FIG. 1A. The device is then ready for continued use in the patient.

In another embodiment, the basket wires can be manipulated by control elements in order to move relative to one another and in order to change or vary the basket configuration for the purpose of more easily entrapping the stone or other material or for some other purpose. The device comprises a medical instrument with an elongated tubular element in which from the distal end of said tubular element is deployed a basket as defined by the presence of three or more wires (each comprised of a proximal end and a distal end) some or all of the distal ends of these basket wires may connected, contiguous, attached, juxtaposed, or nearly juxtaposed to each other. Upon initial deployment or opening of this variable configuration basket, the wires may initially be equidistant, or nearly equidistant from one another with the operator of the device having the ability to manipulate or otherwise alter the spacing and/or configuration of these individual basket wires relative to one another in order for the device to more successfully acquire a stone or other material within a body cavity by creating a wider gap in the spacing between the individual basket wires. Once the desired object has been acquired within the device, the configuration change can be reversed in order to secure the object within the variable configuration basket. Alternatively and/or additionally, in an alternative embodiment, upon initial deployment or opening of the device, the spacing between the wires may be unequal so as to allow for easier acquisition of a stone or other object with the operator of the device effecting a configuration change in the spacing between the wires subsequent to successful acquisition of this stone or other object in order to better secure it. Such new spacing between the wires may be equidistant or nearly equidistant; however, it need not be so. Any change in configuration which better secures the object within the device will do. Additionally and/or alternatively, the basket wires can be manipulated by the control elements in order to move them relative to one another in order to change or vary the basket configuration for the purpose of more easily entrapping the stone or other material or for some other purpose. Configuration changes may occur in one or more steps and may or may not proceed to where some or all of the basket wires are placed in a position of contiguous juxtaposition possibly even forming from a functional standpoint, a two-dimensional loop, sometimes referred to as a snare configuration. Complete configuration change to a flat, two-dimension loop configuration while potentially beneficial is not a necessary feature. Symmetry in motion between each of the basket wires during one or more configuration changes is possible but also not a necessary feature. Individual wires may move in symmetry, uniformly, equally with respect to one another or one or more wires may not move at all relative to each other.

Movement of the basket wires to effect configuration changes may involve movement of both the proximal and distal ends of one wire relative to another of or only either one of these two ends relative to an adjacent wire with the other end maintaining its initial configuration position. If both proximal and distal ends of a particular wire move or shift position, they may move or shift position equally, or one end may move more or less relative to an adjacent wire than the other. The configuration changes may also be effected by a sheathing element, spring loaded hooks, flip levers, effected tension and/or length changes in the wires via a control handle, rings, pincers, micro-electromechanical systems (MEMS), torque, torsion, and/or grooves, notching, or other mechanism along individual basket wires which allow the wires to link up with or connect with one another until the operator chooses to initiate movement of these individual wires relative to one another, allowing them to slide out of the connecting element and to assume new configurations relative to one another.

In an embodiment the variable configuration basket device will be lithotripsy capable such that the individual basket wires are constructed to withstand the forces used to crush and/or shatter a stone or group of stones securely acquired within it. Such a lithotripsy capable embodiment will preferentially be compatible for use with commercially available rescue lithotripsy devices including but not limited to the Soehendra® Lithotripter device. Alternatively, a lithotripsy compatible embodiment of this variable configuration basket can be fitted with its own lithotripsy element to allow for through-the-endoscope lithotripsy. Such a lithotripsy element could take the form of an outer metal sleeve capable of forcefully crushing a secured stone by applying pressure to the basket wires and outer regions of the stone. Alternatively, such a lithotripsy element could take the form of a drill or other object capable of applying a force capable of breaking up an entrapped stone delivered or advanced through the center of the device impacting and crushing the stone at its innermost points within the basket, delivering only modest indirect force to the basket wires. Such an approach to crushing a stone from within the variable configuration basket device such as with a drill may lead to less damage to basket wires or less breakage of the variable configuration basket during lithotripsy as compared to use of an outer metal sleeve to perform such lithotripsy. Any or all of these embodiments are possible and various features may be combined depending on the region, object, and desired end result.

In another embodiment, the variable configuration basket will have an internal space throughout its length capable of allowing for passage of a guidewire from the region of the control element all the way to the distal end of the device and out of the distal end in order to facilitate cannulation of the bile duct and/or pancreatic duct or for some other purposes.

In another embodiment, the variable configuration basket is used for reversibility between configuration changes. For instance, if in an initial stage during acquisition of an object the basket resembles a two-dimensional snare and in the subsequent stage it resembles a basket in which the wires are equidistant or nearly equidistant from each other to secure said object, this variable configuration basket device will therefore preferentially be able to be reversed by the operator of the device back to the snare-like configuration if and when deemed necessary. Such an instance may occur if the variable configuration basket with an entrapped and secured stone were to become impacted at the bottom or distal end of the bile duct as happens with the current art in biliary stone extraction baskets in up to 6% of cases. With current art, such impaction usually requires at the very least emergency rescue lithotripsy but can lead to major surgery as well. Therefore, it is a preferred feature of the variable configuration basket to have the ability to reverse the configuration to allow for an increase in spacing between basket wires should urgent release of an impacted stone become necessary.

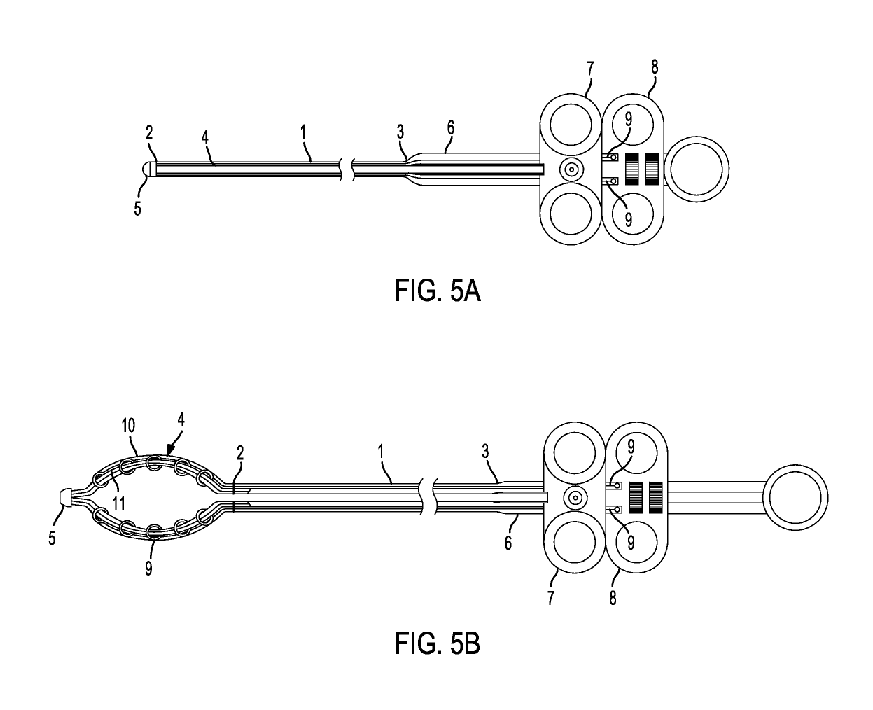

FIGS. 5A and 5B depict embodiments of the device with additional thumbwheel controllers (20, 21) for manipulating the basket configuration. Thumbwheel controllers (20, 21) may be located in or on the slidable handle (8) as shown or may be located on the grip (6) or the handle (7). FIG. 5A depicts the device with the thumbwheel controllers (20, 21) in the handles in the closed position, i.e. where the snare (4) is not deployed. FIG. 5B shows the device with the snare (4) extended or deployed, i.e. in a first snare configuration.

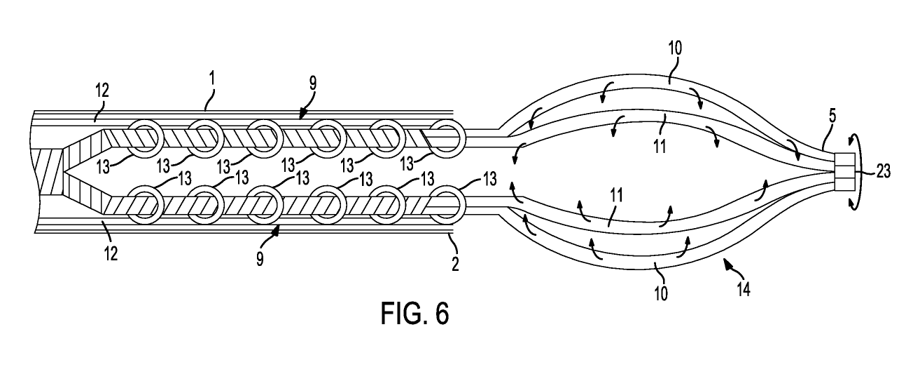

FIG. 6 depicts the variable configuration basket in an open snare basket configuration in which individual basket wires are equidistant or nearly equidistant from each other (14). As shown in FIG. 6 the basket wires (10, 11) may terminate in rotatable pivot point (23). The wires (10, 11) of the variable configuration basket or snare basket device (14) at about, just proximal to, or at their distal ends can fuse into or attach to pivot point (23) just proximal to, or at, the most distal aspect of the variable configuration basket (14), thereby forming the most distal aspect of the variable configuration basket device. The distal end of the variable configuration basket device (14) is not open, but rather the basket wires (10, 11) fit into pivot point (23) either directly (braiding, fusion, etc.) or indirectly (via a mechanical connection point or intervening wire or wires, etc.). Pivot point (23) allows the wires (10, 10a, 11, and 11a) to rotate individually as indicated by the arrows. By utilizing a central pivot point (23), each wire can be rotated individually to configure the basket (14) into a variety of shapes to aid with the retaining of variable shaped and sized objects. It is to be understood, that the invention is not limited to the shown wires (10, 11, 10a, and 11a) but can include more or fewer wires, all or some of which will be held at their distal point by the pivot point (23).

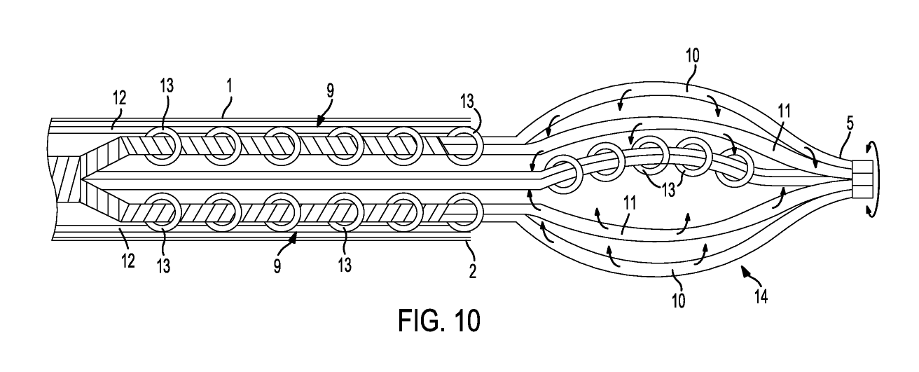

As discussed above, it is understood that the sheath (9) can have various configurations. The configuration shown in, for example, FIG. 6 shows the sheath made of metal with sheath rings (13) supported by a wire sheath backing (12). Alternatively, the sheath can be solid, such as a flexible metal member or flexible plastic or rubber member, or the rings can be partial rings, i.e. not fully closed, or spring loaded hooks or clips. As discussed further with reference to FIG. 10, individual wires may also have individual sheaths such that some or all of the wires (10,11) may be released for deployment at various configurations of this variable configuration basket.

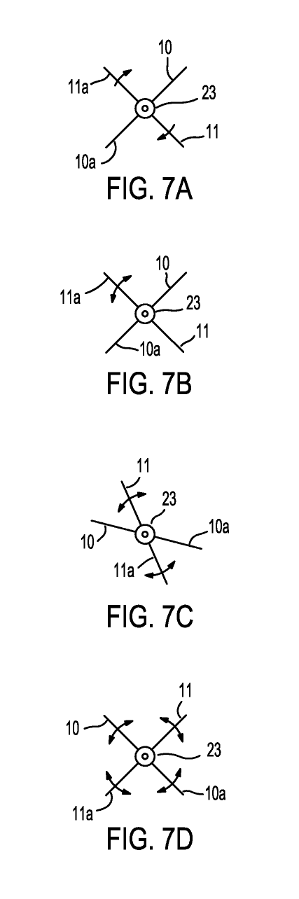

FIGS. 7A-7C depict some possible examples of the individual motion of the wires (10, 10a, 11, and 11a) all rotating from the pivot point (23). FIG. 7A depicts rotation of the wires (10,11) with respect to the pivot point (23), with the wires (10a, 11a) held stationary. As is understood, movement of the wires (10, 11) may be individual or in unison and may be controlled by the thumbwheel controllers (20 or 21) or other similar, handles, wheels, levers, or switches. FIG. 7B depicts an embodiment where only a single wire (11a) is rotatable after deployment with respect to the other three wires (10, 10a, and 11). FIG. 7C depicts an embodiment wherein the wires (11, 11a) are rotatable about the pivot point (23). FIG. 7D depicts an embodiment where all the wires (10, 11, 10a, and 11a) are individually rotatable about the pivot point (23). As noted above rotation of any or all of the wires may be controlled by the thumbwheel controllers (20, 21) or other such devices. It is to be understood that the embodiments depicted in FIG. 7 are simply illustrative and are not intended to limit the combinations of movable and stationary wires. As will be understood by one skilled in the art, numerous combinations and configurations, including the number of wires, the number of stationary, and the number of movable wires may be selected without departing from the present disclosure.

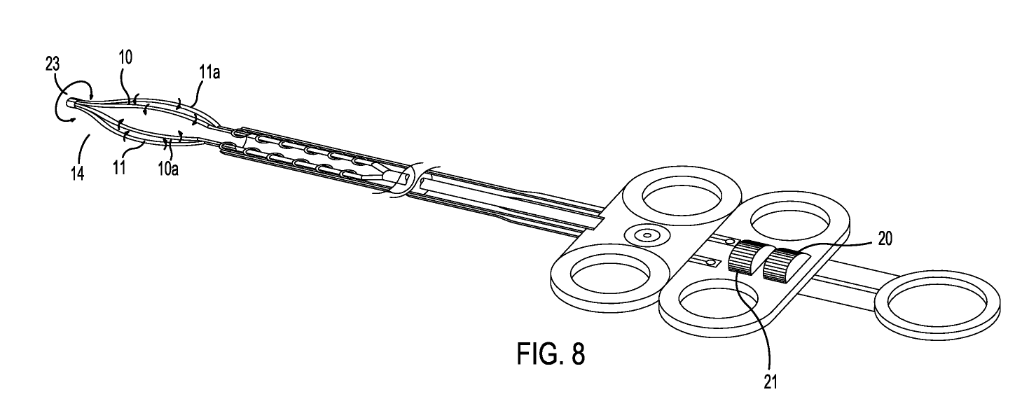

FIG. 8 depicts an embodiment of the device with the sheath withdrawn and the variable configuration basket (14) deployed to a configuration away from a two-dimensional snare-type configuration to one where the basket wires are not equidistant or nearly equidistant from each other. This may represent a final-stage configuration, an intermediate stage of deployment where additional manipulation of the control element could lead to the variable configuration basket assuming a configuration where the individual basket wires will be equidistant or nearly equidistant from each other, or could represent incomplete and ongoing deployment to a configuration where the individual basket wires will be equidistant or nearly equidistant from each other.

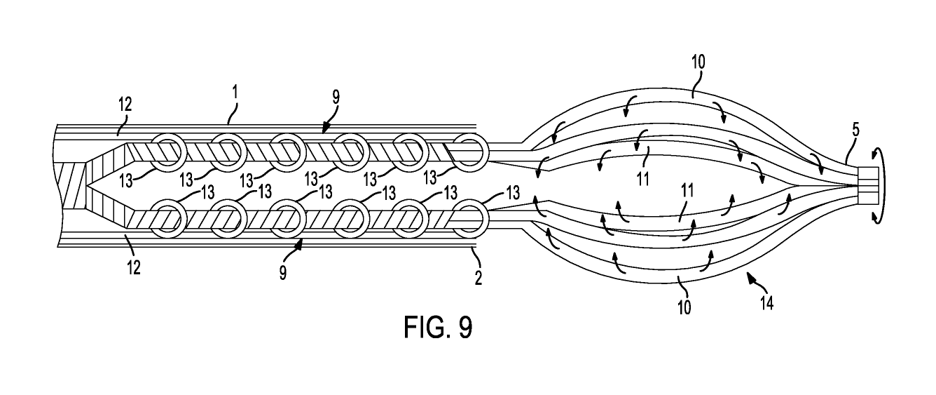

FIG. 9 depicts an embodiment with an additional set of wires (12, 12a) that are connected to the pivot point (23) at the distal end of the snare basket (14). As will be understood by those skilled in the art, the number of wires is not limited to the embodiments shown but may be any configuration, and may be an even or odd amount. The variation in the number of wires connected to pivot point (23) allows for unlimited basket configurations to aid with the retrieval of objects without having to remove or insert additional instruments.

FIG. 10 depicts an embodiment, with separate wires (12, 12a) retained by a separate plurality of sheath rings (24). As depicted, the plurality of sheath rings (13) used to retain the other wires (10, 10a, 11, and 11a) is separately controllable by controller 7 or 8 or any other release and locking mechanism. The additional sheath rings (24) may be controlled by a separate controller (not shown) or an additional release mechanism incorporated into the existing controllers (7, 8). In this embodiment, the wires (12, 12a) may be deployed after the wires (10, 10a, 11, 11a) or at the same time. The additional wires (12, 12a) are individually rotatable and are likewise connected to pivot point (23) at the distal end. The addition of more wires allows for greater flexibility in basket (14) configuration.

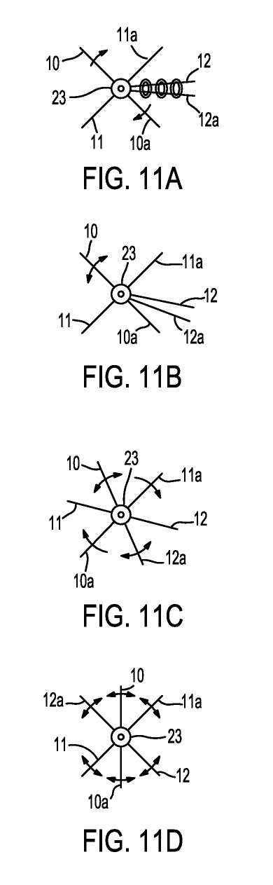

FIGS. 11a-11d depict the rotatibility of the wires (10, 11, 12, 10a, 11a, 12a) as they form basket 14. FIG. 11a, depicts the additional wires (12, 12a) in a retained or sheathed position by sheath rings 24. As will be appreciated, by those skilled in the art, the individual control of the remaining wires (10, 10a, 11, 11a) still allows for configuration of the variable configuration basket (14) even when the additional wires (12, 12a) are not deployed. FIGS. 11b-c depict various possible rotation configurations of the rotatable wires (10, 11, 12, 10a, 11a, 12a). As will be appreciated by those skilled in the art, the combination of rotatable and not rotatable wires is only limited by the number of wires and controllers. (see FIGS. 11b-d).

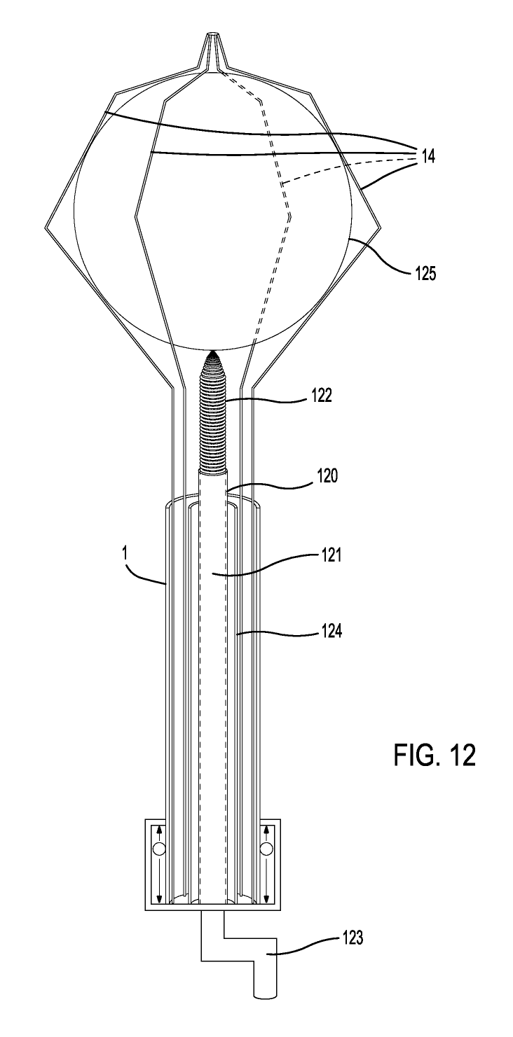

FIG. 12 depicts an embodiment of the variable configuration basket device deployed in a configuration in which the basket wires are equidistant or nearly equidistant with a stone (125) acquired and secured within it. Once the variable configuration basket (14) is in an open/deployed position (i.e., with stone (125) or other object inside of it), the drill will then be extended from the center of the distal end of the elongated tubular element (1), into the proximal end of the variable configuration basket (14) and will be extended or advanced distally toward the distal end of the variable configuration basket. The endoscopic drill contains a shaft (121), a drill bit (122), and a drill drive (123). The endoscopic drill may be used as a mechanical lithotripter to break-up or shatter the entrapped stone (123) at a contact point at the center of an entrapped stone or it may have other modes of operation such as vibrational, ultrasonic, thermal or percussion. As depicted in FIG. 12, the endoscopic drill shaft (121) with bit (122) may be passed through inner tube (120) such that the drill bit (122) contacts the stone (125). The inner tube needs to be large enough to allow the drill shaft (121) and drill bit (122) to pass through, but still be tight enough to prevent lateral movement of the drill within the inner tube (121). As noted in FIG. 12, housed within the outer tube (1) is mid tube (124). The locations of the outer tube (1) and the mid tube (124) create a gap between the outer tube (1) and the mid tube (124) where the basket wires are located. In this manner, the wires will not interfere with the endoscopic drill and may be uninhibitedly controlled, by for example, controllers, thumbwheels, levers, or other manipulation devices from the handles or the proximal end of the device. Furthermore, built into the front of the drill bit (122) may be other sensors (not shown) or stone rupturing devices or heads such as transducers for ultrasonic devices, thermal heads for thermal devices or percussion heads to help rupture and breakup the stone or object.

In another embodiment the variable configuration basket, including but not limited to the snare-basket, may require only a very short or no elongated tubular element at all and may be delivered to the site of interest via other means such as a capsule ingested by the patient.

Presently, wireless capsule endoscopy utilizes a capsule with an imaging mechanism that transmits visual image data to an external recorder. Such devices, however, can only visualize and due not have any therapeutic capability. In an embodiment of the present disclosure, the variable configuration basket, including but not limited to the snare-basket, may be deployed via wireless capsule, such as part of an endoscopic capsule and wirelessly controlled. In such a deployment, The variable configuration basket, including but not limited to the snare-basket will be at or near the site of interest without the need to traverse an elongated tube of an endoscope. Because of the immediate proximity of the basket and snare to the site of interest, the tubular element for deployment need not be via an elongated therapeutic channel of an endoscope. Accordingly, in the present embodiment, the variable configuration basket, including but not limited to the snare-basket may be controlled remotely via any known wireless means without the need for a controller and may not need an elongated tube with a channel to be delivered to the site of interest.

The disclosed embodiments are illustrative of the various ways in which the present invention may be practiced. Other embodiments can be implemented by those skilled in the art without departing from the spirit and scope of the present device.

1. A medical instrument for trapping tissue, polyps, stones and other foreign bodies for retrieval from a body comprising:
a control unit;
an elongated tubular element having an inner channel, wherein the inner channel extends from the control unit to a distal end of the tubular element;
a first set of wires forming a snare configuration, extending from the control unit through the inner channel of the tubular element, and juxtaposed and held in a single loop at the distal end of the inner channel, wherein the single loop can be at least partially retracted into and extended beyond the distal end of the inner channel via the control unit;
at least one wire having a proximal end and a distal end, and forming a basket configuration by converting the snare configuration via the control unit,
wherein the basket as defined by the presence of the first set of wires and the at least one wire comprises some or all of the distal ends of the first set of wires and the at least one basket wire, all of which may be connected, contiguous, attached, juxtaposed, or nearly juxtaposed to each other at a common pivot point.

2. The medical instrument of claim 1, wherein the first set of wires and the at least one basket wire are initially equidistant, or nearly equidistant, or some other configuration of distances from each other such that they can secure a stone or other material acquired from a body cavity when the first set of wires and the at least one basket wire form a basket around said stone or other material.

3. The medical instrument of claim 1, wherein the at least one basket wire can be manipulated by the control unit in order to move relative to the first set of wires in order to change or vary the basket configuration.

4. The medical instrument of claim 1, wherein the control unit contains a controller for controlling the movement of the at least one basket wire.

5. The medical instrument of claim 1 wherein the at least one basket wire and the first set of wires, move in at least one of the following: in symmetry, uniformly, equally with respect to one another, and one or more wires may not move at all relative to each other.

6. The medical instrument of claim 5, wherein configuration changes to the basket configuration may involve movement of both the proximal and distal ends of one basket wire relative to another basket wire.

7. The medical instrument of claim 5, wherein configuration changes to the basket configuration may involve movement of either one of the two ends of the basket wire relative to an adjacent basket wire or the first set of wires, with the other end maintaining its initial configuration position.

8. The medical device of claim 6, wherein the relative movement is an equal move or shift of position or one end may move more or less relative to an adjacent wire than the other.

9. The medical device of claim 2, wherein the configuration changes may be effected by at least one of the following: a sheathing element, a thumbwheel, a spring loaded hook, a flip lever, and effected tension changes in the wires via a control handle, a ring, a pincer, a micro-electromechanical systems (MEMS), a torque, and a torsion.

10. The medical device of claim 2 wherein the configuration change may occur in one or more steps and may or may not proceed to where some or all of the basket wires are placed in a position of contiguous juxtaposition, forming a two-dimensional loop.

11. The medical device of claim 1 further comprising a lithotripter.

12. The medical device of claim 11 wherein the lithotripter is a mechanical lithotripter.

13. The medical device of claim 11 wherein the lithotripter is at least one of the following: a vibrational lithotripter, an ultrasonic lithotripter, a thermal lithotripter and a percussion lithotripter.

14. The medical instrument of claim 4, wherein the controller is a thumbwheel.
